(19) United States
(12) Patent Application Publication
(10) Pub. No.: US 2019/0134346 A1
(43) Pub. Date: May 9, 2019

(54) Vibrating and Sound Baby Mat

(71) Applicants: Steven Lee Kirby, Ranger, TX (US); William Kyle Morrison, Albany, TX (US)

(72) Inventors: Steven Lee Kirby, Ranger, TX (US); William Kyle Morrison, Albany, TX (US)

(21) Appl. No.: 15/805,499

(22) Filed: Nov. 7, 2017

(51) Int. Cl. A61M 21/02

(52) U.S. Cl. CPC A61M 21/02; A61M 2021/0022

(57) ABSTRACT

A vibrating and sound baby mat is an apparatus that effectively comforts an infant. The apparatus includes a plurality of covers, a reinforcing pad, a recorder, a speaker, a vibrating unit, a control panel, and a processing unit. The plurality of covers protects the reinforcing pad, the vibrating unit, and the processing unit from getting damaged. The reinforcing pad upholds and supports an infant. The recorder detects a sound, preferably a heartbeat, and the speaker continuously emits the detected sound. Similarly, the vibrating unit emits vibrations based on the detected sound. In order to better represent the sound, the apparatus may further include an electrocardiogram module and a plurality of electrocardiogram sensors. The control panel engages and controls the recorder, the speaker, the vibrating unit, and the electrocardiogram module. The processing unit allows the recorder, the speaker, the vibrating unit, and the electrocardiogram module to communicate with each other.

### FIELD OF THE INVENTION

The present invention relates generally to baby mats. More specifically, the present invention is vibrating and sound baby mat.

### BACKGROUND OF THE INVENTION

Tending to an infant demands a lot of time, energy, and patience. Learning what exactly comforts an infant, whether the infant simply needs to rest or sleep, or the infant needs to stop crying, is taxing. This is largely due to the fact that infants were being housed and protected within the womb of a mother and are still adjusting to all the sensory inputs in their surrounding environment. A mother, however, cannot constantly be in direct contact or even with her infant throughout the day.

Therefore, it is an objective of the present invention to create an environment for an infant that mirrors that of the womb of a mother. The present invention tends to the senses of touch and hearing. The present invention tends to the sense of primarily touch, distributing vibrations through the baby mat which upholds and envelopes the infant. The vibrations, however, are not just soothing vibrations, but vibrations that mimic the vibrations of the heartbeat of the mother. The present invention also tends to the sense of hearing as the present invention emits sounds that mimic the sound of the heartbeat of the mother. In order to replicate the vibrations and sounds of the mother, the heartbeat of a mother is recorded and sensed by the present invention. The present invention is able to process the input and outputs vibrations and sounds as if the infant is constantly with and pressed against the mother. The present invention also allows a user to decide if an infant is exposed to only vibrations, only sounds, or both vibrations and sounds that mimic a heartbeat.

### BRIEF DESCRIPTION OF THE DRAWINGS

FIG. 1 is a perspective view of the present invention.

FIG. 2 is a perspective view of the present invention without a plurality of covers.

FIG. 3 is a schematic view of the plurality of covers and a reinforcing pad of the present invention.

FIG. 4 is a schematic view of the electronic coupling between the processing unit with the recorder, the speaker, the vibrating unit, and the electrocardiogram module of the present invention.

FIG. 5 is a schematic view of the electrical connection between the power source with the recorder, the speaker, the vibrating unit, and the electrocardiogram module of the present invention.

FIG. 6 is a schematic view of the electrical connection between the power source with the recorder, the speaker, the vibrating unit, and the electrocardiogram module of the present invention, with the power source mounted external to the waterproof cover.

FIG. 7 is a schematic view of the electrical connection between the vibrating unit, the recorder, and the speaker with the vibration button, the recording button, and the volume button of the present invention, respectively.

FIG. 8 is a schematic view of the upper layer, the lower layer, and the flap of the waterproof cover of the present invention, respectively.

### DETAILED DESCRIPTION OF THE INVENTION

All illustrations of the drawings are for the purpose of describing selected versions of the present invention and are not intended to limit the scope of the present invention.

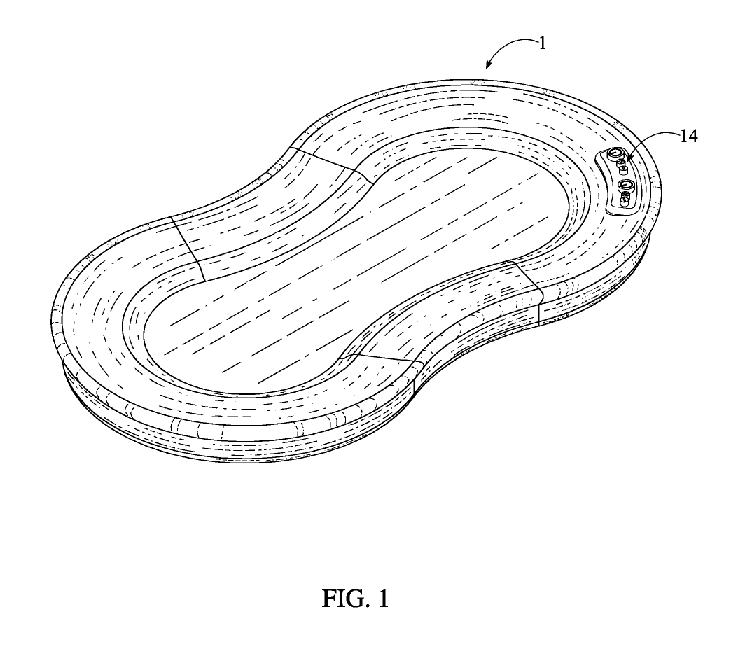

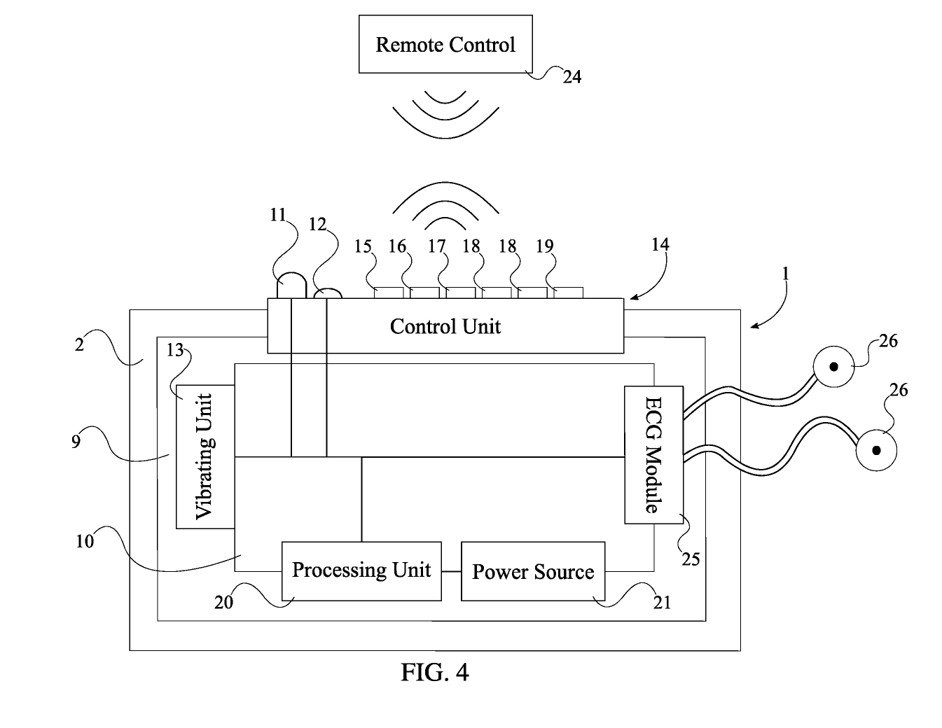

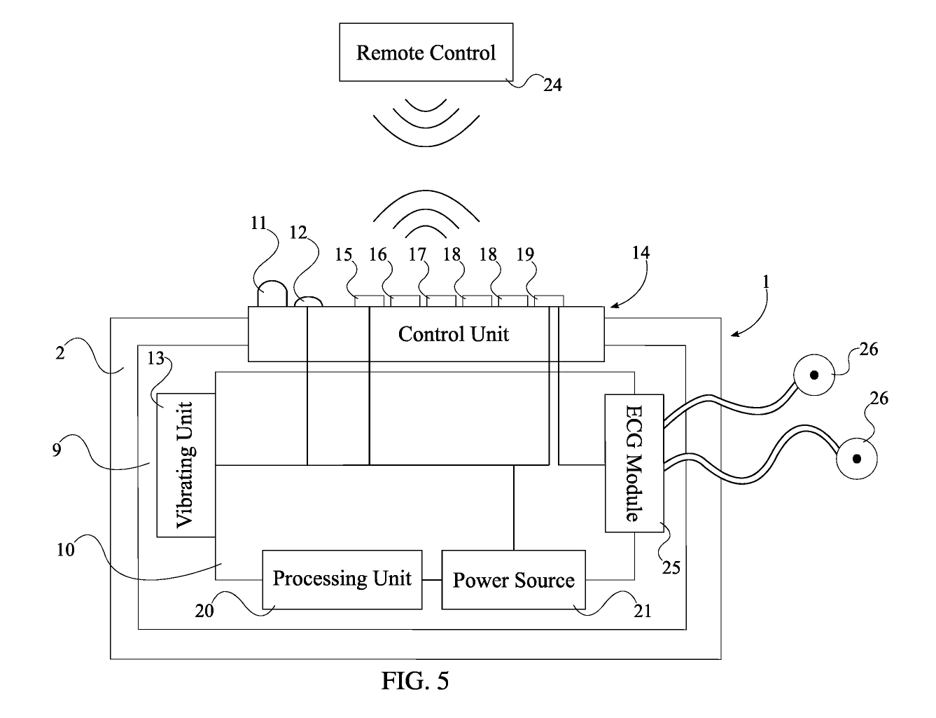

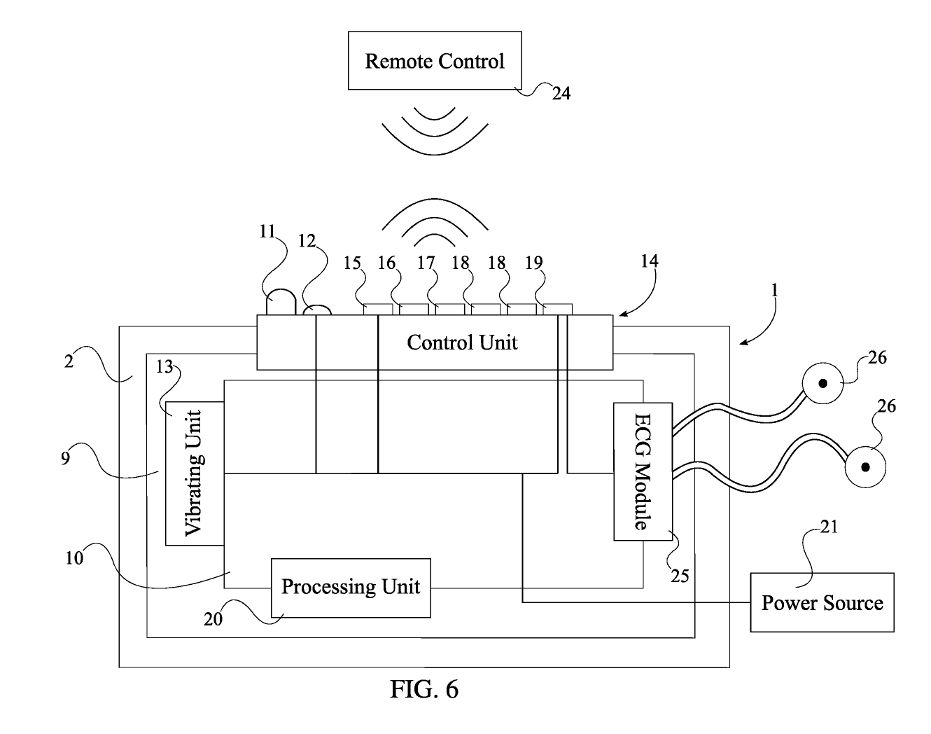

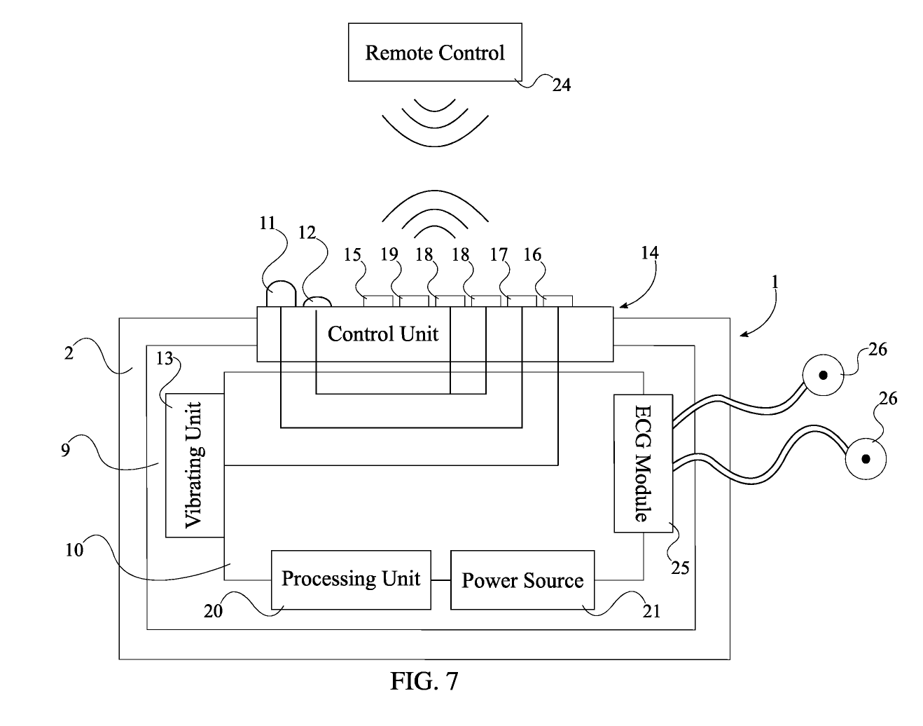

The present invention is a vibrating and sound baby mat that soothes an infant while the infant is resting on the apparatus. The present invention is customizable such that the vibrations and sounds emitted by the present invention mirror that of the heartbeat an individual, preferably the mother. The present invention protects the electronic components contained within or integrated within the apparatus while providing the necessary comfort for an infant. In order to provide a comfortable baby mat that vibrates and emits sounds like the heartbeat of a mother, the present invention comprises a plurality of covers 1, a reinforcing pad 10, a recorder 11, a speaker 12, a vibrating unit 13, a control panel 14, and a processing unit 20, as seen in FIG. 1, FIG. 4, FIG. 5, FIG. 6, and FIG. 7. The plurality of covers 1 protects and cushions the reinforcing pad 10. The plurality of cover also conforms to the structure of the reinforcing pad 10 and surrounds both the vibrating unit 13 and the processing unit 20. The reinforcing pad 10 upholds and surrounds an infant resting on the apparatus. The reinforcing pad 10 is preferably made of low resilience polyurethane foam material which further comforts the infant by adjusting to the contours of the body of the infant. The recorder 11 and speaker 12 captures and emits a sound, respectively. The sound captured and emitted is preferably that of a heartbeat. The vibrating unit 13 emits vibrations which mirror the heartbeat based on the captured sound of the heartbeat. The vibrating unit 13 vibrates the reinforcing pad 10, and consequently the plurality of covers 1. The control panel 14 allows a user to control the recorder 11, the speaker 12, and the vibrating unit 13. The processing unit 20 stores and converts data corresponding to the captured heartbeat and delivers commands to both the speaker 12 and vibrating unit 13.

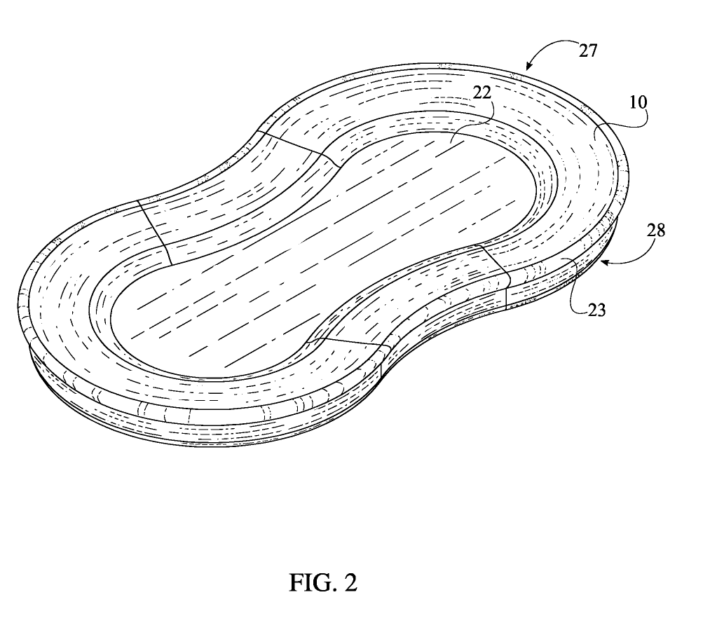

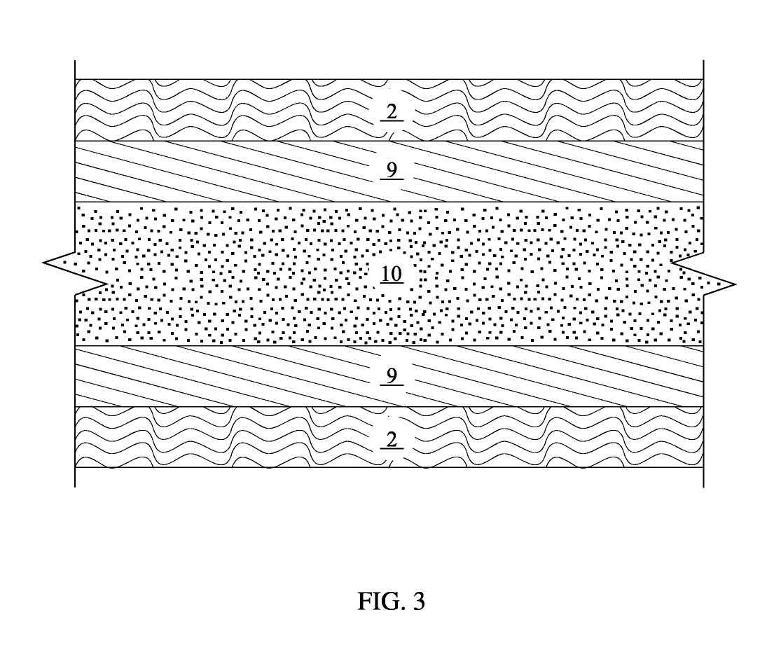

The configuration of the aforementioned components allows the electronic components to be safely housed without minimizing the comfort level of the present invention. The reinforcing pad 10 is positioned within the plurality of covers 1 as to reduce or eliminate any damage to the reinforcing pad 10 caused by the infant, as seen in FIG. 1, FIG. 2, and FIG. 3. Such damage may result from, urine, excrement, vomit, baby food, and so on. The vibrating unit 13 is positioned between the plurality of covers 1 and the reinforcing pad 10. Moreover, the vibrating unit 13 is mounted across the reinforcing pad 10. This arrangement allows the vibrations of the vibrating unit 13 to be evenly distributed across the reinforcing pad 10. Similarly, the processing unit 20 is positioned between the plurality of covers 1 and reinforcing pad 10 such that the processing unit 20 is safely housed and contained within the plurality of covers 1.

In order for the user to access the control panel 14, the control panel 14 is integrated into the plurality of covers 1, illustrated in FIG. 1, FIG. 4, FIG. 5, FIG. 6, and FIG. 7. More specifically, the recorder 11 is integrated into the control panel 14 so that the sound detected by the recorder 11 is not muffled and the recorded sound emitted by the speaker 12 accurately represents original sound. In an alternate embodiment of the present invention, the recorder 11 may comprise a receiving-hub and a removable hand-held recorder 11. The receiving-hub is integrated into the control panel 14 and is electronically coupled to the processing unit 20. The removable hand-held recorder 11 is electrically tethered to the receiving-hub and allows a user to press the removable hand-held recorder to his or her chest, allowing the recorder 11 to accurately detect the sound of a heartbeat. In order for sounds to be detected, stored, and processed effectively, the processing unit 20 is electronically coupled to the recorder 11, the speaker 12, and the vibrating unit 13. In an alternate embodiment of the present invention, a selection of soothing sounds that mimic that of a heartbeat may be stored on the processing unit so that a user may simply choose from the selection instead of recording the actual heartbeat of an individual. It is understood that the control panel further comprises at least one button that accesses the selection and selects a soothing sound from the selection.

Both the electronic components and the reinforcing pad 10 are sufficiently protected, and the comfort of the reinforcing pad 10 is preserved as the plurality of covers 1 comprises a waterproof cover 2 and a cushioning cover 9, seen in FIG. 3, FIG. 4, FIG. 5, FIG. 6, and FIG. 7. The waterproof cover 2 prevents any liquid from coming into contact with the cushioning cover 9. The waterproof cover 2 shields both the reinforcing pad 10 and the electronic components between the plurality of covers 1 and the reinforcing pad 10. The cushioning cover 9 absorbs the impact of the weight and the movements of an infant that is resting on the apparatus. The cushioning cover 9 is accessible through the waterproof cover 2. Preferably, the cushioning cover 9 surrounds the reinforcing pad 10 along the surfaces an infant may come into contact with while resting on the present invention. Furthermore, the cushioning cover 9 is easily removable such that the vibrating unit 13 and the processing unit 20 are easily accessible. The reinforcing pad 10 is positioned within the cushioning cover 9 so that the infant is safely upheld and supported, while further being comforted by the cushioning cover 9. The cushioning cover 9 is positioned within the waterproof cover 2 in order to prevent the cushioning cover 9, and consequently the reinforcing pad 10, from getting damaged. Furthermore, the vibrating unit 13 and the processing unit 20 are positioned between the cushioning cover 9 and the reinforcing pad 10, shielding the vibrating unit 13 and the processing unit 20 from any damage.

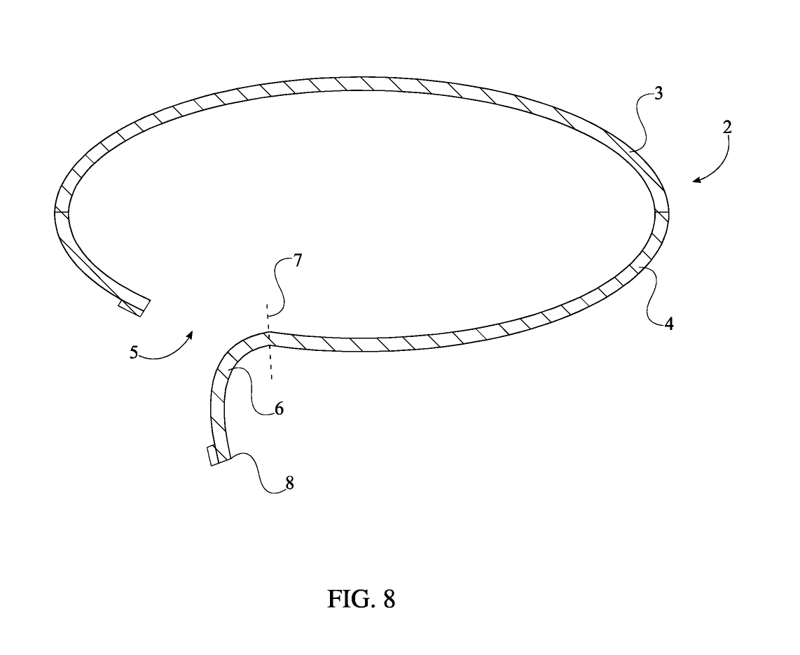

In order to access the electronic components housed between the cushioning cover 9 and the reinforcing pad 10, the waterproof cover 2 comprises an upper layer 3, a lower layer 4, an opening 5, and a flap 6, as shown in the schematic view of FIG. 8. The upper layer 3 and the lower layer 4 effectively surround the cushioning layer. The opening 5 allows a user to access the cushioning cover 9, the reinforcing pad 10, the vibrating unit 13, and the processing unit 20. The flap 6 seals the opening 5 in order to conceal and secure the components contained within the waterproof cover 2. The waterproof cover 2 surrounds the entirety of the cushioning cover 9 as the upper layer 3 and the lower layer 4 are perimetrically connected to each other. The opening 5 traverses through the lower layer 4 so that the infant resting on the apparatus is not able to accidentally access the components within the waterproof cover 2. The flap 6 is hingedly connected to the upper layer 3 along a stationary edge 7 of the flap 6 and is positioned adjacent to the opening 5. A pivoting edge 8 of the flap 6 is positioned opposite the stationary edge 7 of the flap 6 across the flap 6. This arrangement allows the flap 6 to expose and seal the opening 5 while remaining connected to the lower layer 4. A fastener may be integrated along the pivoting edge 8 and a corresponding edge of the lower layer 4. The fastener detachably attaches the pivoting edge 8 of the flap 6 to the corresponding edge of the lower layer 4 in order to effectively expose and seal the opening 5. The fastener may include, but is not limited to, velcro, snaps, and zippers.

As seen in FIG. 4, FIG. 5, FIG. 6, and FIG. 7, the present invention further comprises a power source 21 so that the recorder 11, the speaker 12, the vibrating unit 13, the control panel 14, and the processing unit 20 may receive power in order to operate. In order for a user to control the electronic components of the present invention, the control panel 14 comprises a power button 15, a vibration button 16, a recording button 17, and a volume button 18. The power button 15 controls the power delivery from the power source 21 to the recorder 11, the speaker 12, and the vibrating unit 13. The vibration button 16, the recording button 17, and the volume button 18 controls features, specifically power delivery, of the vibrating unit 13, the recorder 11, and the speaker 12, respectively. The vibration button 16 preferably turns on and off the vibrating unit 13, as the vibrating unit 13 continuously vibrates until the vibration button 16 is engaged. In an alternate embodiment of the present invention, the vibration button 16 may also control the strength of vibrations emitted from the vibrating unit 13. The recording button 17 preferably activates and deactivates the recorder 11 such the user may initiate and terminate the recording of a sound, preferably a heartbeat, within a given area of the recorder 11. The volume button 18 preferably increases and decreases the loudness of the sound emitted from the speaker 12. In the preferred embodiment of the present invention, the volume button 18 is preferably a couple of buttons such that a button of the couple of button increases the loudness of the speaker 12 and the other button of the couple of buttons decreases the loudness of the speaker 12. In an alternate embodiment of the present invention, the control panel 14 may comprise a speaker power button that turns on and turns off the power of the speaker 12 as the speaker 12continuously emits sound. The power button 15 is able to turn on and turn off the vibrating unit 13, the recorder 11, and the speaker 12 simultaneously.

In the preferred embodiment of the present invention, the power source 21 is positioned between the plurality of covers 1 and the reinforcing pad 10, seen in FIG. 4, FIG. 5, FIG. 6, and FIG. 7. thereby limiting any contact with dangerous hazards near and around the infant. The power source 21 of the preferred embodiment is a replaceable power source 21 or a rechargeable power source 21. In an alternate embodiment of the present invention, the power source 21 is mounted external to the waterproof cover 2, seen in FIG. 6. The power source 21 may therefore be a portable battery, an outlet, and so on.

The user may easily control and access each button as the power button 15, the vibration button 16, the recording button 17, and the volume button 18 are distributed across the control panel 14, shown in FIG. 4, FIG. 5, FIG. 6, and FIG. 7. Moreover, the user may easily control and access the recorder 11 and the speaker 12 as the recorder 11 and the speaker 12 are both integrated into the control panel 14. The power source 21 is electrically connected to the speaker 12, the vibrating unit 13, and the power button 15, thereby allowing the power button 15 to simultaneously turn on and turn off the speaker 12 and the vibrating unit 13 through the power button 15. The vibration button 16 is electronically connected to the vibrating unit 13, the recording button 17 is electronically connected to the recorder 11, and the volume button 18 is electronically connected to the speaker 12. These connections allow the recorder 11, the vibrating unit 13, the speaker 12 to be controlled by the corresponding buttons.

The present invention not only comforts an infant but supports and protects the infant. The preferred embodiment of the present invention further comprises a depression 22 and a supporting rim 23, seen in FIG. 2. The depression 22 hugs and surrounds an infant resting on the apparatus and, and the supporting rim 23 serves as a barrier that physically alerts in infant that he or she is about to fall from the apparatus as the infant comes into contact with the supporting rim 23. In this preferred embodiment, the reinforcing pad 10 comprises a first face 27 and a second face 28. The first face 27 and the second face 28 both comprise a peanut-like shape. This peanut-like shape contours to the overall shape of an infant and serves to maintain the orientation of the infant within the depression 22 while also allowing for the limbs to move freely. The first face 27 is positioned opposite the second face 28 about the reinforcing pad 10. The depression 22 is integrated into the first face 27, and the supporting rim 23 is perimetrically fixed to the first face 27 so that an infant resting within the depression 22 may freely move within the depression 22. More specifically, the supporting rim 23 is positioned around the depression 22 so that the infant is made aware if he or she is about to fall from the apparatus

An alternate embodiment of the present invention further comprises a remote controller 24, illustrated in FIG. 4, FIG. 5, FIG. 6, and FIG. 7. The remote controller 24 allows a user to manage and engage the electronic components that are electronically connected to the control panel 14. More specifically, the remote controller 24 is communicably coupled to the control panel 14. This communication allows a user to turn on, turn off, and adjust the settings of the electronic components which are electrically connected to the control panel 14 from a distance.

An alternate embodiment of the present invention further comprises an electrocardiogram (ECG) module 25 and a plurality of ECG sensors 26, also seen in FIG. 4, FIG. 5, FIG. 6, and FIG. 7. The ECG module 25 processes the vibrations of the heartbeat detected by the ECG module 25 and delivers the data to the processing unit 20. The plurality of ECG sensors 26 detects the vibrations of a heartbeat. Various embodiments of the plurality of ECG sensors 26 may comprise an adhesive layer that attaches the plurality of ECG sensors 26 to the chest of an individual. In this alternate embodiment, the control panel 14 further comprises an ECG power button 19, which turns on and off the ECG module 25 and, consequently, the plurality of ECG sensors 26. The ECG module 25 is positioned between the plurality of covers 1 and the reinforcing pad 10, similar to that of the vibrating unit 13 and the processing unit 20. Moreover, as the ECG module 25 is not directly engaged during each use and may get damaged from any liquids, and so on, the ECG module 25 is effectively protected and stored in this arrangement. The plurality of ECG sensors 26 is positioned external to the plurality of covers 1 so that the plurality of ECG sensors 26 may directly contact the chest of an individual. In order for the vibrations of the heartbeat to be detected and processed by the ECG module 25, the plurality of ECG sensors 26 is electrically tethered to the ECG module 25. In order for the power button 15 to simultaneously turn on and turn off the power of all the electronic components of the present invention, the power source 21 is electrically connected to the ECG module 25 through the ECG power button 19. The vibrating unit 13 is able to emit vibrations that mirror the heartbeat of the corresponding individual as the processing unit 20 is electronically coupled to the ECG module 25, thereby delivering the data from the ECG module 25 to the vibrating unit 13.

Although the invention has been explained in relation to its preferred embodiment, it is to be understood that many other possible modifications and variations can be made without departing from the spirit and scope of the invention as hereinafter claimed.

What is claimed is:

1. A vibrating and sound baby mat comprises:
a plurality of covers;
a reinforcing pad;
a recorder;
a speaker;
a vibrating unit;
a control panel;
a processing unit;
the reinforcing pad being positioned within the plurality of covers;
the vibrating unit being positioned between the plurality of covers and the reinforcing pad;
the processing unit being positioned between the plurality of covers and the reinforcing pad;
the vibrating unit being mounted across the reinforcing pad;
the control panel being integrated into the plurality of covers;
the processing unit being electronically coupled to the recorder, the speaker, and the vibrating unit; and,
the processing unit being vibrationally connected to the recorder and the vibrating unit.

2. The vibrating and sound baby mat as claimed in claim 1 comprises:
the plurality of covers comprises a waterproof cover and a cushioning cover;
the reinforcing pad being positioned within the cushioning cover;
the cushioning cover being positioned within the waterproof cover;
the vibrating unit being positioned between the cushioning layer and the reinforcing pad; and,
the processing unit being positioned between the cushioning layer and the reinforcing pad.

3. The vibrating and sound baby mat as claimed in claim 2 comprises:
the waterproof cover comprises an upper layer, a lower layer, an opening, and a flap;
the upper layer and the lower layer being perimetrically connected to each other;
the opening traversing through the lower layer;
the flap being hingedly connected to the upper layer along a stationary edge of the flap;
the flap being positioned adjacent to the opening; and,
a pivoting edge of the flap being positioned opposite the stationary edge of the flap across the flap.

4. The vibrating and sound baby mat as claimed in claim 1 comprises:
a power source;
the control panel comprises a power button, a vibration button, a recording button, and a volume button;
the recorder and the speaker being integrated into the control panel;
the power button, the vibration button, the recording button, and the volume button being distributed across the control panel;
the power source being electrically connected to the speaker, the vibration unit, and the power button;
the vibration button being electronically connected to the vibrating unit;
the recording button being electronically connected to the recorder; and,
the volume button being electronically connected to the speaker.

5. The vibrating and sound baby mat as claimed in claim 4 comprises:
the power source being positioned between the plurality of covers and the reinforcing pad.

6. The vibrating and sound baby mat as claimed in claim 4 comprises:
the power source being mounted external to the waterproof cover.

7. The vibrating and sound baby mat as claimed in claim 1 comprises:
a depression;
a supporting rim;
the reinforcing pad comprises a first face and a second face;
the first face being positioned opposite the second face about the reinforcing pad;
the depression being integrated into the first face;
the supporting rim being perimetrically fixed to the first face; and,
the supporting rim being positioned around the depression.

8. The vibrating and sound baby mat as claimed in claim 1 comprises:
a remote controller; and,
a remote controller being communicably coupled to the control panel.

9. The vibrating and sound baby mat as claimed in claim 1, wherein the reinforcing pad is made of a low resilience polyurethane foam material.

10. The vibrating and sound baby mat as claimed in claim 1 comprises:
a power source;
an electrocardiogram (ECG) module;
a plurality of ECG sensors;
the control panel comprises a ECG power button;
the ECG module being positioned between the plurality of covers and the reinforcing pad;
the plurality of ECG sensors being tethered to the ECG module;
the plurality of ECG sensors being positioned external to the plurality of covers;
the ECG power button being connected to the control panel;
the power source being electrically connected to the ECG module;
the ECG power button being electrically connected to the ECG module and the plurality of sensors;
the plurality of ECG sensors being electrically connected to the ECG module;
the processing unit being electronically coupled to the ECG module; and,
the processing unit being vibrationally connected to the ECG module and the vibrating unit.
